(19) United States
(12) Patent Application Publication
(10) Pub. No.: US 2022/0039866 A1
(43) Pub. Date: Feb. 10, 2022

(54) SELECTING A PROSTHESIS AND IDENTIFYING A LANDING ZONE FOR IMPLANTATION OF THE PROSTHESIS

(71) Applicant: Medtronic Vascular, Inc., Santa Rosa, CA (US)

(72) Inventors: Mostafa Toloui, Mounds View, MN (US); Mark A. Hoff, Santa Rosa, CA (US); Geoffrey Orth, Santa Rosa, CA (US)

(73) Assignee: Medtronic Vascular, Inc., Santa Rose, CA (US)

(21) Appl. No.: 17/391,306

(22) Filed: Aug. 2, 2021

Related U.S. Application Data
(60) Provisional application No. 63/060,774, filed on Aug. 4, 2020.

(51) Int. Cl. A61B 34/10; G16H 10/60; G16H 20/40; G16H 50/30; A61F 2/24; A61B 34/20

(52) U.S. Cl. CPC A61B 34/10; G16H 10/60; G16H 20/40; A61B 2034/101; A61F 2/2427; A61B 34/20; G16H 50/30

(57) ABSTRACT

An example method includes receiving, via at least one processor, anatomical measurements of a lumen of a patient. The method includes performing, via the at least one processor, a geometrical fit analysis based on the anatomical measurements to identify potential prostheses to be implanted in the lumen and an optimal implantation landing zone within the lumen for at least one of the potential prostheses, wherein the geometrical fit analysis includes comparing a geometry of the lumen, including shape factors for the lumen, to geometries of a plurality of candidate prostheses at a plurality of potential implant deployment positions within the lumen. The method includes performing, via the at least one processor, a biomechanical interaction analysis to select one of the identified potential prostheses based on a risk of migration within the lumen of each of the identified potential prostheses. The method includes outputting, via the at least one processor, an indication of the selected prosthesis and the landing zone for the selected prosthesis.

### CROSS REFERENCE TO RELATED APPLICATIONS

This Non-Provisional patent application claims the benefit of the filing date of U.S. Provisional Patent Application Ser. No. 63/060,774, filed Aug. 4, 2020, entitled “Selecting A Prosthesis And Identifying A Landing Zone For Implantation Of The Prosthesis,” which is herein incorporated by reference.

### FIELD

The present technology is generally related to a system and method for automatically selecting a prosthesis, and automatically identifying a landing zone for implantation of the selected prosthesis.

### BACKGROUND

It is important to select an appropriately configured prosthesis, such as a prosthetic heart valve, because if the prosthetic heart valve does not fit properly, the prosthetic heart valve may migrate, leak or cause other problems. In order to select an appropriately sized prosthetic heart valve, the size, shape, topography, compliance and other physical parameters of a vessel lumen may be assessed. In some circumstances, an exhaustive image collection and image measurements may be analyzed for selecting a prosthetic heart valve configured to fit a patient's particular anatomy.

Various devices are also available for internally determining the size and other physical parameters of an internal orifice or lumen. Such devices can include an expandable member, such as a balloon, capable of expanding to contact tissue and collect information relating to physical parameters of the tissue proximate the expandable member.

Patient screening for a prosthesis, such as a prosthetic heart valve, can be challenging due to the anatomical complexities of the patient population. Some screening processes may be costly, time-consuming, subjective, and not sufficiently predictive.

The present disclosure provides improvements associated with the related art.

### SUMMARY

The techniques of this disclosure generally relate to assessment of a suitable prosthesis, such as a suitable prosthetic heart valve, for a patient, including identifying a landing zone for implantation and displaying the landing zone for a clinician in one or more views.

In one aspect, the present disclosure provides a method, which includes receiving, via at least one processor, anatomical measurements of a lumen of a patient. The method includes performing, via the at least one processor, a geometrical fit analysis based on the anatomical measurements to identify potential prostheses to be implanted in the lumen and an optimal implantation landing zone within the lumen for at least one of the potential prostheses, wherein the geometrical fit analysis includes comparing a geometry of the lumen, including shape factors for the lumen, to geometries of a plurality of candidate prostheses at a plurality of potential implant deployment positions within the lumen. The method includes performing, via the at least one processor, a biomechanical interaction analysis to select one of the identified potential prostheses based on a risk of migration within the lumen of each of the identified potential prostheses. The method includes outputting, via the at least one processor, an indication of the selected prosthesis and the landing zone for the selected prosthesis.

In another aspect, the present disclosure provides a method of identifying a prosthesis for implantation and a landing zone for implantation of the prosthesis within a patient's anatomy at an implantation site. The method includes receiving, via at least one processor, a three-dimensional model of the implantation site. The method includes analyzing, via the at least one processor, for each of a plurality of potential prostheses, a plurality of potential prosthesis deployment positions and axis orientations relative to the three-dimensional model. The method includes identifying, via the at least one processor, the prosthesis for implantation from the plurality of potential prostheses based on the analyzing. The method includes identifying, via the at least one processor, a landing zone at the implantation site for the identified prosthesis. The method includes generating, via the at least one processor, a display illustrating the landing zone in a preoperative image.

In another aspect, the present disclosure provides a non-transitory computer-readable storage medium storing instructions that, when executed by a processor, cause the processor to perform a geometrical fit analysis based on anatomical measurements at a prosthesis implant site of a patient to identify potential prostheses to be implanted at the implant site, wherein the geometrical fit analysis includes comparing an anatomical geometry at the implant site to geometries of a plurality of candidate prostheses at a plurality of potential implant deployment positions at the implant site; perform a probabilistic mechanical force analysis to determine a risk of failure of each of the identified potential prostheses; and output a recommendation identifying one of the potential prostheses based on the probabilistic mechanical force analysis.

In another aspect, the present disclosure provides an electronic prosthesis analysis tool, which includes a memory to store a plurality of different design concepts for a prosthesis, and a processor to perform a probabilistic mechanical force analysis on the plurality of different design concepts to determine prosthesis failure risk information for each of the design concepts and identify a best one of the design concepts based at least in part on the prosthesis failure risk information.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram illustrating a computing system for identifying a patient specific prosthesis and a patient specific landing zone according to one embodiment.

FIG. 2 is a flow diagram illustrating a method of identifying a patient specific prosthesis and a patient specific landing zone according to one embodiment.

FIGS. 3A and 3B are diagrams illustrating additional details regarding the method of identifying a patient specific prosthesis and a patient specific landing zone according to one embodiment.

FIGS. 4A and 4B are diagrams illustrating additional details regarding the geometrical fit analysis performed in the method shown in FIG. 2 according to one embodiment.

FIGS. 5A-5C are diagrams illustrating a geometrical device fit evaluation validation against clinical experience involving implant tests in patient-specific 3D printed models of patients from a TPV clinical trial.

FIGS. 6A and 6B are diagrams illustrating additional details regarding the biomechanical interaction analysis performed in the method shown in FIG. 2 according to one embodiment.

FIGS. 7A and 7B are diagrams illustrating (7A) a back-calculation methodology used to evaluate the model's prediction against ten device clinical cases; and (7B) the evaluation results.

FIGS. 8A and 8B are diagrams illustrating a schematic representation of a methodology to characterize the contribution of anatomical shape factors to device retention force according to one embodiment.

FIGS. 9A-9C are diagrams illustrating a migration finite element analysis (FEA) model for a curved configuration.

FIG. 10A is a diagram illustrating FEA migration onset for a first criteria according to one embodiment.

FIG. 10B is a diagram illustrating FEA migration onset for a second criteria according to one embodiment.

FIG. 11A is a schematic illustration of anatomical shape creation.

FIG. 11B is a diagram illustrating a sample of resulted 3D printed models.

FIG. 12A shows a sample of implant test and grading of device. fit/apposition.

FIG. 12B shows device OS % quantitative evaluation (e.g., using Materialize Mimics) for tubular structure (D=38 mm, C=0.02 mm⁻¹ and E=0.4).

### DETAILED DESCRIPTION

I. Introduction

Examples disclosed herein are directed to an automated prosthetic device patient screening method with intraoperative visualization. Some examples may be directed to an automated native right ventricular outflow tract (RVOT) transcatheter pulmonary valve (TPV) patient screening method with intraoperative visualization. Some prosthetic heart valve devices are designed to be implanted within the main pulmonary artery (PA) (e.g., between RVOT and PA bifurcation). It is noteworthy that there is a large anatomical variation in size and shape in native RVOT space. In addition, unlike Transcatheter Aortic Valve Replacement (TAVR) and Transcatheter Mitral Valve Replacement (TMVR) spaces, the valve does not have a well-defined landing zone. Although some examples are described in the context of prosthetic heart valves, techniques described herein may be applied to any type of prosthesis, and may be used to provide an automatic selection of an appropriately sized prosthetic device, and to provide visual guidance to an implanting physician, such as displaying an optimal landing zone for the selected device.

In some examples, a preoperative prosthetic heart valve patient screening method automatically evaluates candidacy of a patient for a prosthetic heart valve device, and provides a patient specific landing zone guide for implant. In some examples, a patient specific landing zone is identified based on a geometrical fit analysis and a biomechanical interaction analysis. Examples of the data-driven method evaluate the device-anatomy interaction based on both geometry and force balance using preoperative data (e.g., medical imaging, etc.). The method takes into account the anatomical size and shape, physiological pressures, and device design and manufacturing variations.

Examples disclosed herein improve outcomes and patient safety via enabling: (1) health care providers to easily perform a multiphase device-fit evaluation for a patient; (2) recommend the patient's candidacy for a prosthetic device; (3) recommend an appropriate prosthetic device to implant; (4) and recommend an implant location/zone. In some examples, the recommended landing zone may be communicated in both magnetic resonance (MR)/computed tomography (CT) based simulated intraoperative fluoroscopic images and overlaid on live intraoperative fluoroscopic images for intraoperative visuals/guidance purposes. The images output by embodiments of the present disclosure provide physicians with an easy to understand representation, and enhance the intra-operative visualization experience.

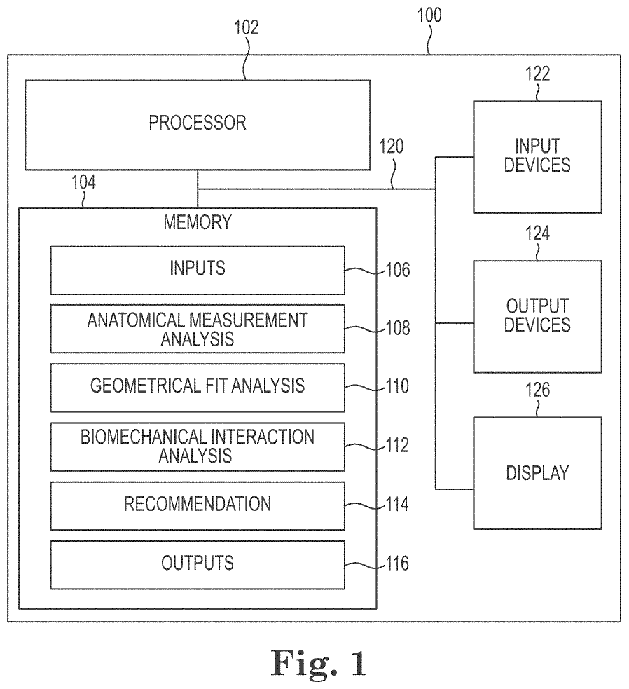

FIG. 1 is a block diagram illustrating a computing system 100 for identifying a patient specific prosthesis and a patient specific landing zone according to one embodiment. System 100 includes processor 102, memory 104, input devices 122, output devices 124, and display 126. Processor 102, memory 104, input devices 122, output devices 124, and display 126 are communicatively coupled to each other through communication link 120.

Input devices 122 include a keyboard, mouse, data ports, stylus or digital pen, and/or other suitable devices for inputting information into system 100. Output devices 124 include speakers, data ports, and/or other suitable devices for outputting information from system 100. Display 126 may be any type of display device that displays information to a user of system 100.

Processor 102 includes a central processing unit (CPU) or another suitable processor. In an example, memory 104 stores machine readable instructions executed by processor 102 for operating system 100. Memory 104 includes any suitable combination of volatile and/or non-volatile memory, such as combinations of Random-Access Memory (RAM), Read-Only Memory (ROM), flash memory, and/or other suitable memory. These are examples of non-transitory computer readable media (e.g., non-transitory computer-readable storage media storing computer-executable instructions that when executed by at least one processor cause the at least one processor to perform a method). The memory 104 is non-transitory in the sense that it does not encompass a transitory signal but instead is made up of at least one memory component to store machine executable instructions for performing techniques described herein.

Memory 104 stores inputs 106, anatomical measurement analysis module 108, geometrical fit analysis module 110, biomechanical interaction analysis module 112, recommendation module 114, and outputs 116. Processor 102 executes instructions of modules 108, 110, 112, and 114 to perform techniques described herein based on inputs 106 to generate outputs 116. In some examples, the inputs 106 include preoperative images for a patient. Module 108 performs an anatomical measurement analysis. Module 110 performs a geometrical fit analysis. Module 112 performs a biomechanical interaction analysis, which involves device-anatomy interaction biomechanics (and migration of the prosthesis). Based on the various analyses by the modules 108, 110, and 112, the recommendation module 114 generates outputs 116, which may include an identification of a patient specific prosthesis, and a patient specific landing zone for the identified prosthesis.

In one example, the various subcomponents or elements of the system 100 may be embodied in a plurality of different systems, where different modules may be grouped or distributed across the plurality of different systems. To achieve its desired functionality, system 100 may include various hardware components. Among these hardware components may be a number of processing devices, a number of data storage devices, a number of peripheral device adapters, and a number of network adapters. These hardware components may be interconnected through the use of a number of busses and/or network connections. The processing devices may include a hardware architecture to retrieve executable code from the data storage devices and execute the executable code. The executable code may, when executed by the processing devices, cause the processing devices to implement at least some of the functionality disclosed herein.

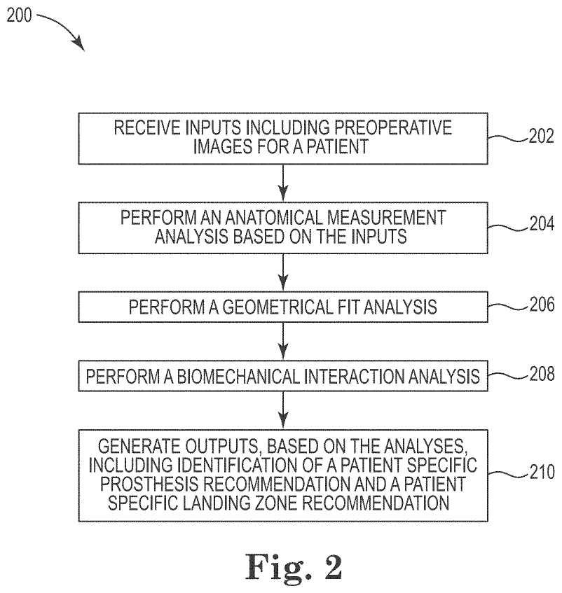

FIG. 2 is a flow diagram illustrating a method 200 of identifying a patient specific prosthesis and a patient specific landing zone according to one embodiment. In some examples, computing system 100 (FIG. 1) is configured to perform method 200. At 202, the method 200 includes receiving inputs including preoperative images for a patient. At 204, the method 200 includes performing an anatomical measurement analysis based on the preoperative images. The anatomical measurement analysis at 204 may be performed by module 108 in system 100. At 206, the method 200 includes performing a geometrical fit analysis. The geometrical fit analysis at 206 may be performed by module 110 in system 100. At 208, the method 200 includes performing a biomechanical interaction analysis. The biomechanical interaction analysis at 208 may involve a probabilistic mechanical force system analysis to evaluate device-anatomy interaction biomechanics (and migration of the device). The biomechanical interaction analysis at 208 may be performed by module 112 in system 100. At 210, the method 200 includes generating outputs, based on the analyses at 204, 206, and 208, wherein the outputs include identification of a patient specific prosthesis recommendation, and a patient specific landing zone recommendation for the identified prosthesis. The generation of outputs at 210 may be performed by module 114 in system 100. The proposed landing zone may be illustrated on simulated intra-op fluoroscopic images using pre-op CTs. This is aimed at providing a good estimate on starting intra-op angiographic angles to potentially improve ease of use and reduce the X-ray exposure for patients.

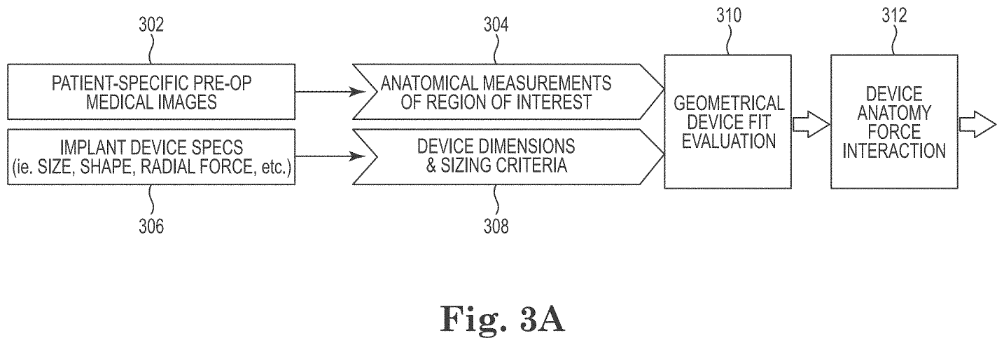

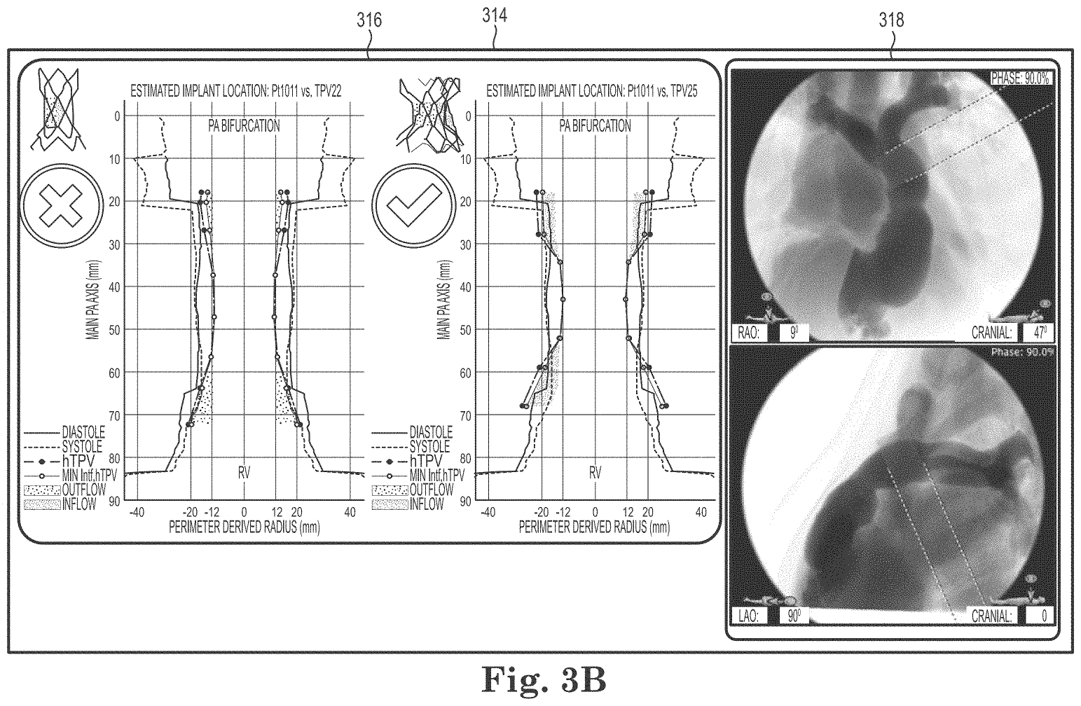

FIGS. 3A and 3B are diagrams illustrating additional details regarding the method 200 of identifying a patient specific prosthesis and a patient specific landing zone according to one embodiment. As shown in FIG. 3A, patient-specific pre-op medical images 302 are used to determine anatomical measurements of a region of interest 304. Implant device specifications (e.g., size, shape, radial force, etc.) 306 are used to determine device dimensions and sizing criteria 208. The anatomical measurements 304 and device dimensions and size criteria are provided to geometrical device fit evaluation module 310, which provides geometrical device fit information to device-anatomy force interaction module 312. Module 310 is an example of module 110 (FIG. 1), and module 312 is an example of module 112 (FIG. 1). Device-anatomy force interaction module 312 provide device-anatomy force interaction information to recommendation module 314, which is shown in FIG. 3B. Module 314 is an example of recommendation module 114 (FIG. 1). In an example, based on the information received from module 312, module 314 provides the following recommendations: (1) a device selection recommendation (i.e., which device to implant); (2) implant location/depth recommendation (i.e., where to implant); and (3) cautions/risk factors (i.e., watch-outs). FIG. 3B also shows an example of implementation for a TPV valve, where a selection of which device to implant is made at 316, and a determination of where to implant the device is made at 318.

The anatomical measurement analysis, geometrical fit analysis, and biomechanical interaction analysis performed in the method 200 shown in FIGS. 2 and 3 are described in further detail below. Additional information is also described in U.S. Pat. No. 10,322,000, entitled “SIZING CATHETERS, METHODS OF SIZING COMPLEX ANATOMIES AND METHODS OF SELECTING A PROSTHESIS FOR IMPLANTATION”, filed Apr. 5, 2018, and issued Jun. 18, 2019, which is hereby incorporated by reference herein.

II. Anatomical Measurement Analysis

This section describes an anatomical measurement analysis, which may be performed by module 108 in computing system 100 (FIG. 1).

A lumen for receiving a prosthesis may have a large patient-to-patient variation in size and shape, which results in a complex anatomic screening process. For example, the delivery device landing zone may be anatomy-dependent and may vary patient to patient, and multiple measurements along the length of the lumen may be used to assess device fit. In some examples, imaging may be performed for potential patient candidates, and the images may be subjected to detailed measurements of the theoretical prosthesis landing zone in multiple phases of cardiac cycle (e.g. both end-systole (30%) and end-diastole (90%) states). Centerline-based geometrical measurements may be extracted in both phases of interest corresponding to maximum and minimum lumen size. These measurements may be performed across the anatomical centerline, i.e. taking cross-sectional measurements along the length of the lumen.

III. Geometrical Fit Analysis

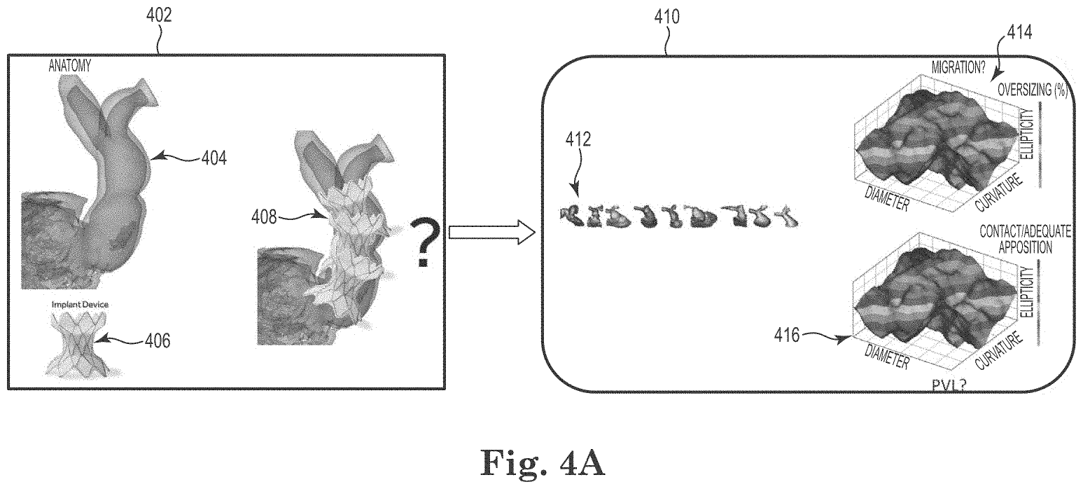

This section describes a geometrical fit analysis, which may be performed by module 110 in computing system 100 (FIG. 1). FIGS. 4A and 4B are diagrams illustrating additional details regarding the geometrical fit analysis performed in the method 200 shown in FIGS. 2 and 3 according to one embodiment. As shown in FIG. 4A at 402, all possible implant locations/scenarios of an implant device 406 in a patient's anatomy 404 are examined. Various possible locations/scenarios are shown at 408. At 410, a data-driven (informed by finite element analysis (FEA)/test) size and shape process results in a three-dimensional (3D) fit map. There is a variable anatomical shape within the target population. The process may involve examining the anatomical space with respect to shape factors of interest (e.g., curvature and ellipticity). Anatomies of various sizes and shapes are shown at 412. Corresponding data distributions may be extracted. FEA/test may be implemented to create a database (shape and size), and device fit maps 414 and 416 may be generated. Machine learning may be used to find the transfer function: A support vector machine (SVM) with Gaussian Kernel decision boundary may be used as a robust and large margin classifier, and it may be constructed and trained on the data. Predicted device fit for a TPV example implementation is shown at 418 in FIG. 4B. Three sample implant scenarios within the landing zone are shown at 420, and two sample implant scenarios outside the landing zone are shown at 422.

The geometrical fit analysis module 110 interfaces with anatomical measurements (e.g., provided by a user or from measurement software). In some embodiments, centerline-based measurements may be inputted into the geometrical fit analysis module 110, where generally the anatomical measurements are compared to those of manufacturer specified device requirements, and a patient's candidacy for the device or prosthesis based on anatomical size & shape parameters is evaluated.

Given the large anatomical variation within the target population, some prosthetic device treatments currently involve an extensive pre-operative patient screening/selection process. Some screening processes face major challenges and limitations such as: (1) labor and time-intensive; (2) expensive; (3) inter-user variability (subjective); and (4) insufficient predictivity. These limitations make such screening processes not scalable for a commercial product. Notably, performing test implants in patient-specific 3D printed replicas to better predict the device fit, can be one of the most challenging parts of this process. The importance of patient-specific 3D printing stems from the fact that the device fit in native lumen anatomies is a function of both shape and size (i.e. morphology and dimensionality), and a perimeter plot (PP) approach may only capture the effect of anatomical size. A perimeter plot-based sizing approach may show an acceptable outflow-inflow apposition, but a stent graft implantation test in patient-specific 3D printed models may show acceptable and unacceptable device fit (e.g., there may be a significant device-anatomy gap at, for example, the inflow section of the device, indicating inadequate oversizing and a potential for migration and/or leakage).

An anatomical geometrical profile (e.g. perimeter, curvature, ellipticity etc. profiles) may be imported/inputted into the geometrical fit analysis module 110, where oversizing ratios (OS %) may be calculated for a plurality of implant locations along the corresponding lumen axis. These OS % values are calculated at critical sections of the device specified by prosthesis manufacturers. For example, for outflow (OF) and inflow (IF) oversizing ratios (OS %) and sizes are calculated for each implant scenario. The geometrical fit analysis module 110 first computes these OS % values based on the size profiles. For example, the difference between the prosthesis and anatomical perimeter at every grid point (ΔPi). Through dividing these ΔPi values with those of the corresponding device size, the geometrical fit analysis module calculates the net sum OS %. This computation may be implemented only for points where anatomical perimeter is smaller than that of the device. This value is, then, divided by its corresponding length (i.e., the length of contact between the device and the anatomy).

Then, to account for shape and topology of the anatomy, the geometrical fit analysis module 110 recalculates the OS % and device fit based on a data-driven algorithm created by experiments (e.g. 3D print anatomy generation followed by device fit CT-scan analysis) and simulations (e.g. 3D CAD anatomy generation followed by device fit FEA analysis) of device fit in variable representative anatomies of the target population. These representative anatomies include both patient-specific and/or artificially generated anatomies.

To create the data-driven algorithm, device performance in different anatomical shapes with respect to both fit (e.g., absence of significant gap as an indicator to prevent leakage) and OS % (as an indicator to prevent migration) have been studied. The fit ranking demonstrates inverse relation to both size and shape factors, i.e. the fit ranking declines with increase in ellipticity (E), curvature (C) and size (D). However, the OS % shows a direct relation with ellipticity (E) while being inversely related to changes in size (D) and curvature (C).

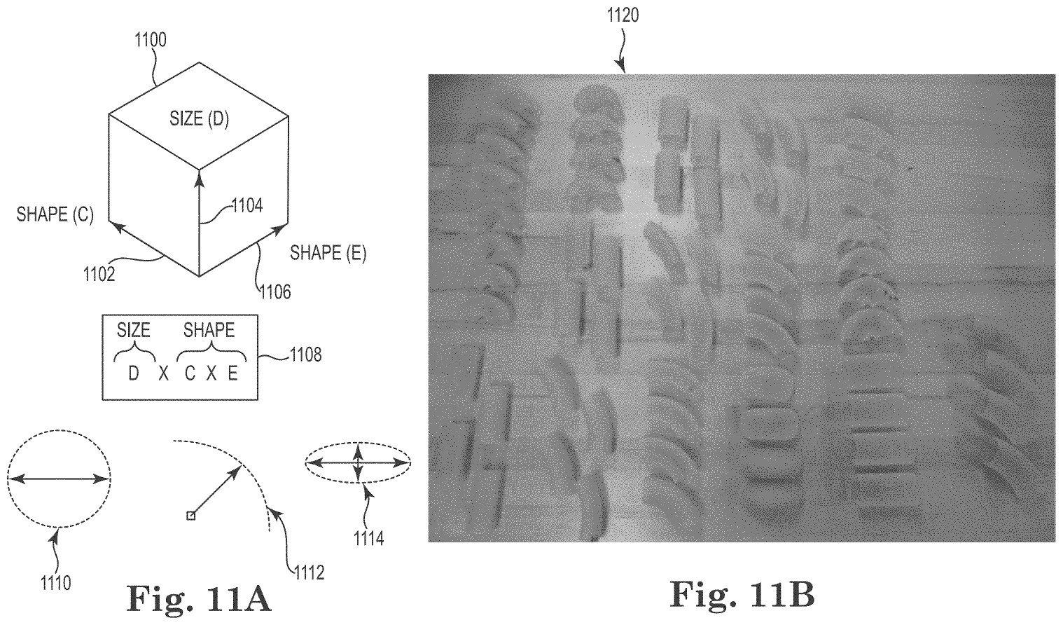

To create artificial anatomies: Pre-op CT data for a prosthetic device may be quantitatively characterized by the geometrical fit analysis module 110 using device dimensions. A corresponding distribution of extracted geometrical factors may then be sampled into equally spaced values, for example, for ellipticity: E=0.2, 0.4, 0.6, 0.8 and for radius of curvature: C=26 mm, 38 mm, 50 mm, 62 mm, 98 mm and Go (straight). In addition to these values of ellipticity and curvature, separate sizes (in the form of perimeter derived diameter) of D=29 mm, 32 mm, 35 mm, 38 mm, 43 mm and 48 mm, for example, may be used to generate all possible combinations of tubular structures (x=D×E×C). This process is illustrated in FIG. 11A. FIG. 11A is a schematic illustration of anatomical shape 1100 creation. FIG. 11A shows shape curvature (C) 1102, shape ellipticity (E) 1106, and size (diameter (D)) 1104, which may be multiplied together as indicated at 1108 to indicate all possible combinations. An example diameter (D) is shown at 1110. An example curvature (C) is shown at 1112. An example ellipticity (E) is shown at 1114. These geometrical combinations were then designed using SolidWorks as CAD models, and 3D printed into rigid tubular models using Vero Clear material (FIG. 11B). FIG. 11B is a diagram illustrating a sample of resulted 3D printed models 1120.

Ellipticity may be defined as shown in the following Equation I:

$$E = \sqrt{\frac{R {1}^{2} - R {2}^{2}}{R {1}^{2}}} \qquad Equation I$$

Where: R1 and R2 represent largest and smallest radiuses, respectively.

Higher ellipticity values represents a more oval and less circular form cross-section and a circle has E=0.

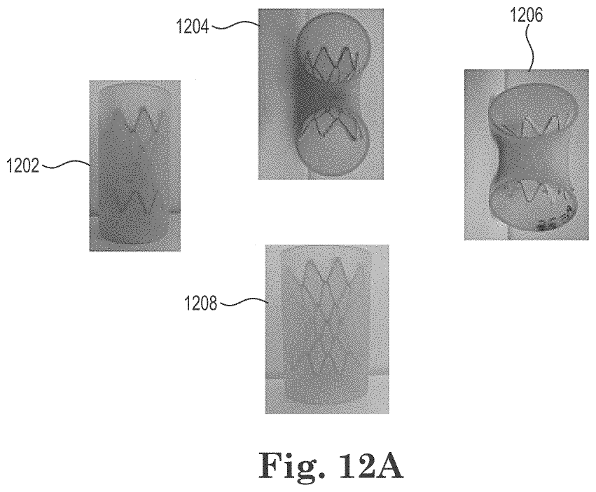

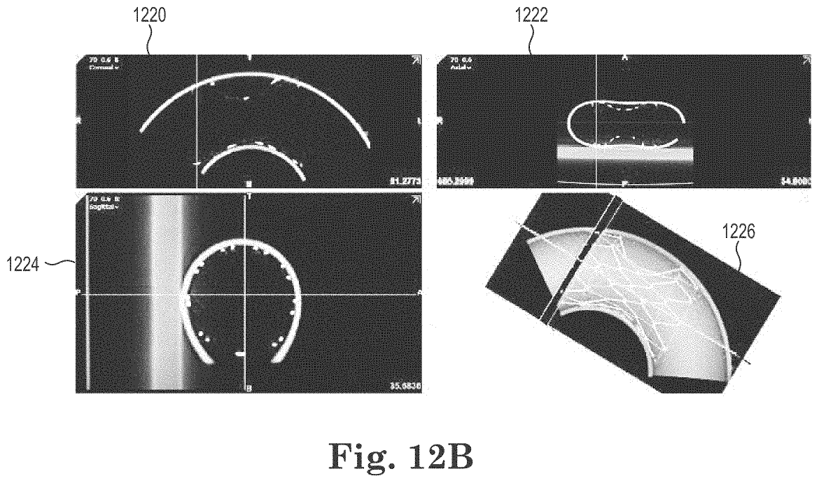

These geometrical combinations may then be designed as CAD models, and 3D printed into rigid or flexible tubular models. Flexibility of the models could be adjusted to those of the anatomical compliance. The models may then be subject to an implant test experimentally or via simulation. For example, in experimental approach, as FIGS. 12A and 12B show: (1) device fit on both inflow and outflow sections may be ranked by physicians (e.g., unacceptable, borderline, and acceptable); (2) and, in addition to this subject labeling/evaluation, the implanted devices may be CT-scanned, and the CT-scans may be analyzed to extract device perimeters at two inflow nodes and two outflow nodes. FIG. 12A shows a sample of implant test and grading of device fit/apposition. Example 1202, with D=38 mm, C=0, and E=0.2, has good apposition. Example 1204, with D=38 mm, C=0.04, and E=0.2, has bad apposition. Example 1206, with D=38 mm, C=0.04, and E=0.8, has bad apposition. Example 1208, with D=38 mm, C=0, and E=0.8, has borderline apposition. FIG. 12B shows device OS % quantitative evaluation (e.g., using Materialize Mimics) represented by images 1220, 1222, 1224, and 1226 for tubular structure (D=38 mm, C=0.02 mm⁻¹ and E=0.4).

Computer simulation via CAD and FEA modeling could be used, as an alternative or in addition to the experimental approach, for this purpose.

These perimeters may be used to calculate the OS % ratios of implanted devices for all these implants as shown in the following Equation II:

$$OS \% = \frac{\begin{matrix}Device Fully Expanded Perimeter - \\ CT Measured Perimeter\end{matrix}}{Device Fully Expanded Perimeter} . \qquad Equation II$$

Subsequently, the device OS % and inflow OS % may be calculated as an average of calculated OS % values of the two outflow and inflow nodes, respectively.

The x (i.e. D, C, E)→y and x (i.e. D, C, E)→OS % values may be used to train a two multivariate transfer functions for both fit and OS %. Specifically, the exported D, C, and E from outflow and inflow sections of the device for any implant scenario/location from the geometrical fit analysis module may be imported into corresponding transfer functions to determine the corresponding estimates of fit (y) and oversizing (OS %) for inflow and outflow sections, respectively. The trained predictive models (i.e., the calculated hyperplane or decision boundaries) may then be evaluated against patients screened for a prosthetic device. The predicted fit from the algorithm may be compared against the outcome of the screening committee, where implanting physicians evaluated the device fit using the corresponding cases' patient-specific 3D-printed models.

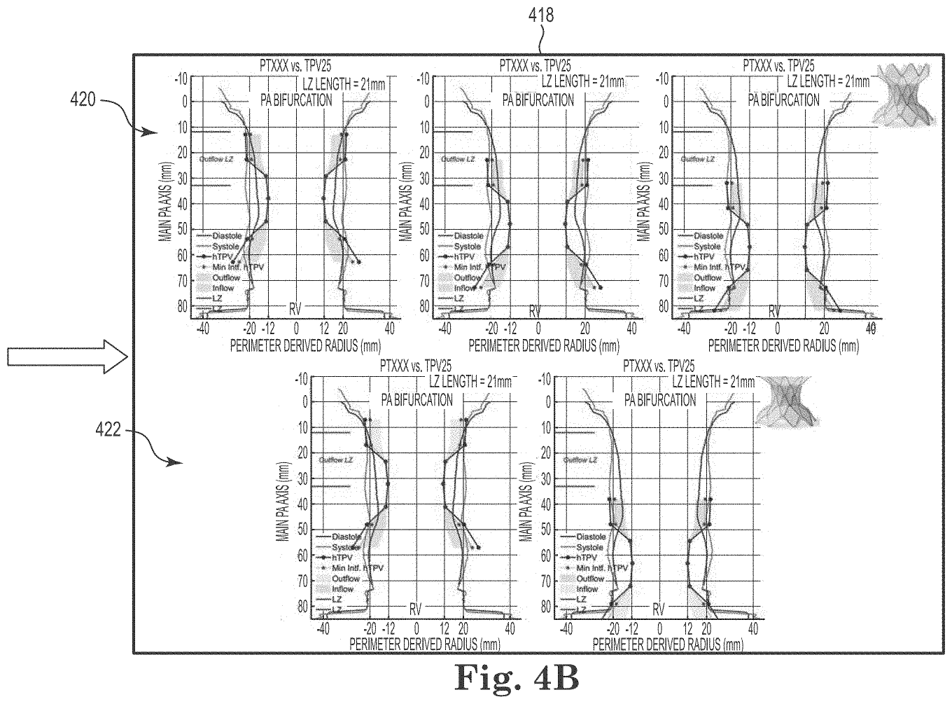

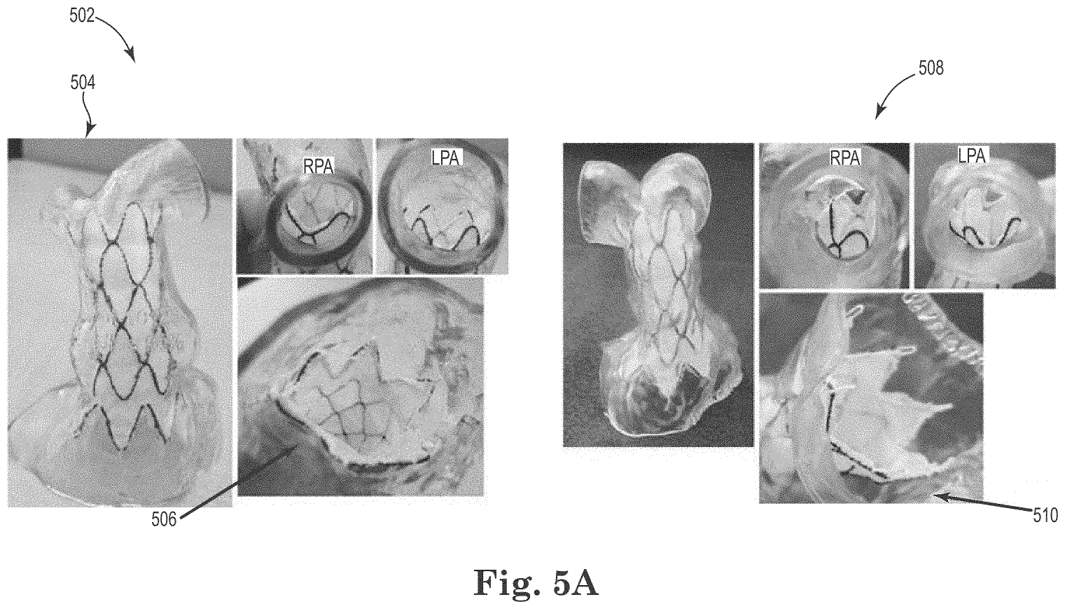

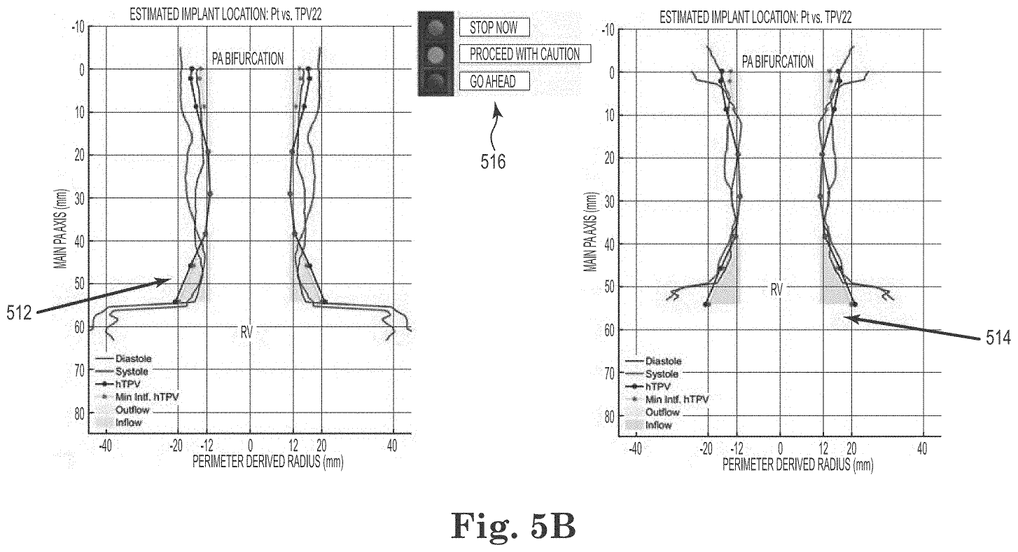

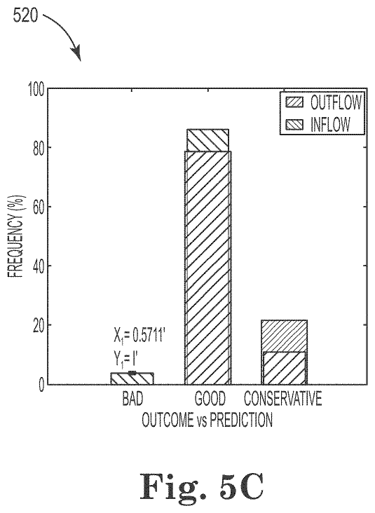

The uncertainty of this methodology may be evaluated through comparison between computational estimates of OS % and the effect on acceptable/unacceptable device fit decision making versus those of experimentally measured values (e.g., from corresponding CT scanned patient-specific models). FIGS. 5A-5C are diagrams illustrating a geometrical device fit evaluation validation against clinical experience involving implant tests in patient-specific 3D printed models of patients from a TPV clinical trial. As shown in FIG. 5A, example 504 in the process 502 shows adequate apposition, as indicated at 506. Example 508 in the process 502 shows an observed gap and inadequate apposition, as indicated at 510. In the histogram representation 520 shown in FIG. 5C, bad represents cases where the computational model predicted acceptable fit while experiments showed borderline or unacceptable fit. Good represents those cases where both models predicted the same (acceptable/unacceptable). Finally, the conservative prediction bucket includes those cases where the computational model identified acceptable implant scenarios as unacceptable. Being focused on consumer risk, this algorithm shows only less than 4% uncertainty ( 1/28 implant scenarios).

Then, the geometrical fit analysis module 110 computes the landing zone (LZ) and apposition appropriateness based on finding sections of the lumen, where both the inflow and outflow OS % ratios are above or equal to the minimum required OS % ratios, which are specified by prosthesis manufacturers. FIG. 5B shows two estimated implant location examples 512 and 514 that are predicted by the model and include an easy to understand traffic light base illustration 516. The landing zone identifies the axial extend of the zone/region within the lumen to target device outflow implantation, which predicts adequate OS % on both inflow and outflow of the device. Notably, the landing zone calculation is based on perimeter-based comparison of the inputs from both the anatomical measurements and the prosthesis dimensions and characteristics.

Finally, the geometrical fit analysis module 110 then computes corresponding landing zones for each of (or at least a plurality of) the prosthesis candidates, which was scanned in 1 mm intervals, for example.

The geometrical fit analysis module 110 may estimate inflow and outflow anatomy-device length of contact and oversizing index for every possible implant scenario along the lumen based on the anatomical size and shape input values. The length of contact between the device and the anatomy may be estimated from the perimeter profiles of the device and the anatomy. Specifically, the geometrical fit analysis module 110 may calculate the axial length of the region where the anatomical perimeter profile is smaller than that of the prosthesis, in minimum interference stage, at the inflow and outflow for a given implant scenario. The oversizing estimate may be represented by area between the anatomical perimeter profile and that of the prosthesis in fully expanded phase at inflow and outflow sections.

Examples disclosed herein assess the anatomical adequacy of patients for a prosthesis using patient-specific anatomical measurements from pre-op imaging (e.g., CT). In some examples, a centerline-based perimeter measurement is graphically plotted (e.g., perimeter plot [PP]) in both phases corresponding to maximum and minimum lumen size. The PP approach provides a graphical means for comparing anatomy perimeter to device perimeter along the entire length of the potential implant site. It allows evaluation of predicted oversizing or interference fit at the inflow and outflow sections of the device at various implant positions. Some examples account for shape factors, such as curvature and ellipticity, in addition to a device-anatomy size comparison (e.g., using perimeter) in prediction of the device-anatomy fit.

Some examples disclosed herein provide recommendations and insight for an implanting physician implanter. The geometrical fit analysis module 110 (e.g., fit analysis software) is a tool that computes the device apposition fit based on the inputs from both the anatomical measurements (e.g., provided to the software by imaging analysts) and the screening criteria or device design specs (e.g., device dimensions and characteristics (e.g., with respect to leakage and migration performance)). In addition, implanting physicians may further evaluate and confirm the device fit.

IV. Biomechanical Interaction Analysis

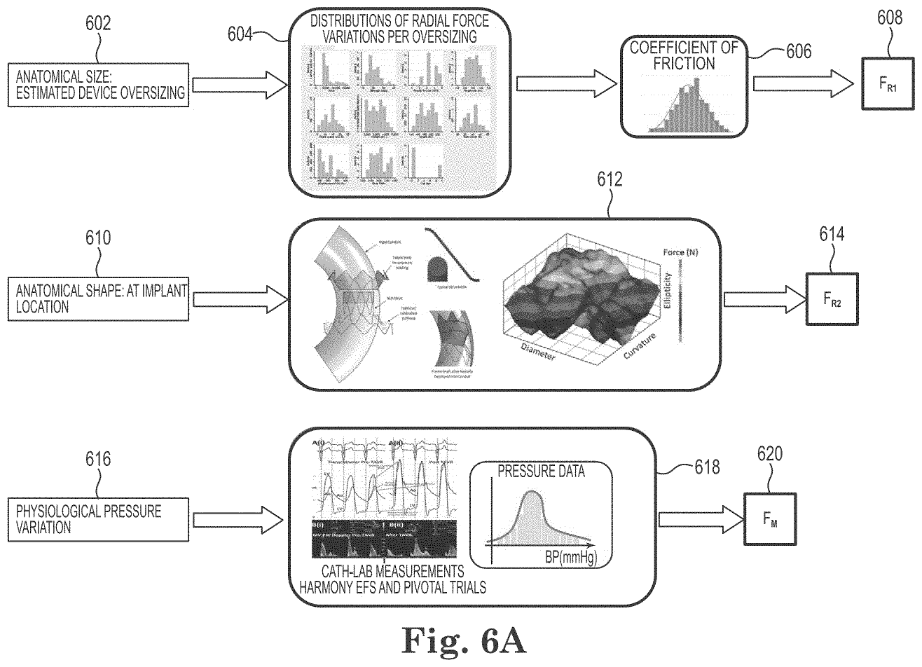

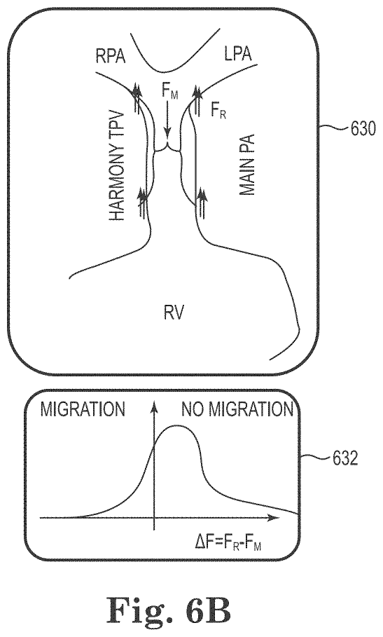

This section describes a biomechanical interaction analysis, which may be performed by module 112 in computing system 100 (FIG. 1). FIGS. 6A and 6B are diagrams illustrating additional details regarding the biomechanical interaction analysis performed in the method 200 shown in FIGS. 2 and 3 according to one embodiment. In some embodiments, the biomechanical interaction analysis uses a first principle based probabilistic device-anatomy force interaction model.

The biomechanical interaction analysis module 112 provides a predictive model for prosthesis migration, and provides a tool: (1) to evaluate the risk of migration for different design concepts; and (2) inform future design or patient screening process to improve the outcomes.

Some prosthetic devices primarily rely on compression on both inflow and outflow sections to generate normal force on the device-anatomy interface to keep the device in place. Therefore, the biomechanical interaction analysis module 112 uses a screening process capable of estimating the oversizing ratios in a pre-operative setting (specifically outputs of the geometrical fit module). The biomechanical interaction analysis module 112 evaluates the suitability of patients for prosthetic devices based on these estimates calculated from pre-operative CTA examination of patients.

FIGS. 6A and 6B show a system of concurrent migration versus resistive forces acting on a prosthetic heart valve during the migration's critical cardiac phase (i.e. diastolic). As shown in FIG. 6A, the migration force (F_{M}) 620 is characterized with dislodging forces imposed on prosthetic device (for example, in the case pulmonic prosthetic valve the downward force resulted from diastolic back pressure is one of those dislodging forces). In addition, the resistance force is broken into multiple major components (i.e. F_{R1} 608, F_{R2} 614 and potentially additional forces). The first component F_{R1} represents the friction-based resistive force, which is mainly associated with the radial force of the valve frame resulted from anatomical size factors. The second component F_{R2} accounts for the retention force contribution from anatomical shape features such as curvature, ellipticity, etc. As shown in FIG. 6A, anatomical shape at implant location information 610 is used at 612 in a data-driven (informed by FEA/test) shape process to generate a 3D force map to determine F_{R2} 614. Additional components may, for example, embody the effect of more complex factors such as device-tissue embedding. These components may be investigated and characterized in succession/step by step process through a statistical model. The statistical model may be built using the data from screening analysis on pre-operative CTAs, intra-operative hemodynamic pressure measurements, device characterization test results such as but not limited to chronic outward force (COF) characterization (test and FEA simulation), and device-tissue interaction test. The radial force or COF may be measured at various diameters. Finite element analysis (FEA) may be used to simulate the COF over the complete sizing range.

A multivariate (e.g., device radial force (COF), anatomical size, coefficient of friction, physiological pressure, anatomical shape (e.g., curvature and ellipticity) and anatomical compliance model (See FIG. 6A) may be developed, which starts with an anatomical size distribution. As shown in FIG. 6A, anatomical size and estimated device oversizing information 602 is used at 604 to determine device manufacturing variability (e.g., informed by manufacturing variability of radial force, COF crimp test), including distributions of radial force variations per oversizing. The selected size allows selection of the corresponding COF distribution at 606 and estimation of outflow area. From the COF distribution, a COF value may be randomly picked. This COF is then multiplied with a coefficient of friction value randomly selected from its corresponding distribution (from test). This results in the COF contribution to F_{R1} 608. The tissue-device surface friction variation at 606 may be informed by a pull test in anatomically relevant tissue conduits, or back-calculation use FEA to match post-op CTs. Physiological pressure variation information 616 is used at 618 to generate a physiological pressure distribution. A pressure value may be randomly selected from the physiological pressure distribution. This value indicates the migration force after multiplication with calculated outflow area: F_{M} 620.

This process may be repeated for many iterations and from the calculated F_{R1} and F_{M} values the ΔF=F_{R1}−F_{M} distribution is formed, where the area under ΔF>0 indicates the risk associated with migration. FIG. 6B shows an example of implementation 630 in a TPV space. The migration force, F_{M}, is equal to ΔP×A. The resistance force, F_{R}, is equal to F_{R1}+F_{R2}+ . . . .

FIG. 6B also shows a probabilistic prediction 632 on risk of migration, which indicates an X % risk of migration for this implant location/scenario/case.

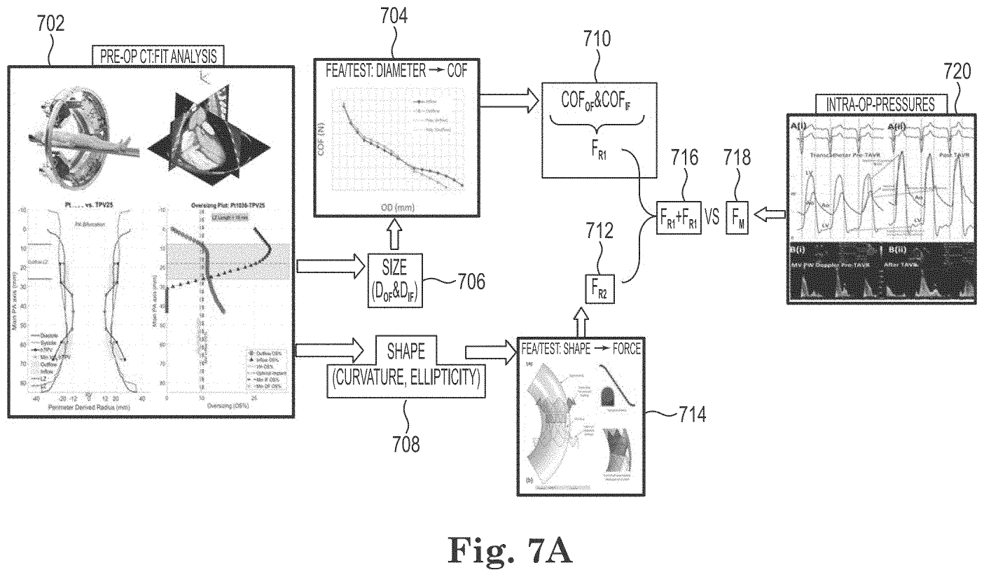

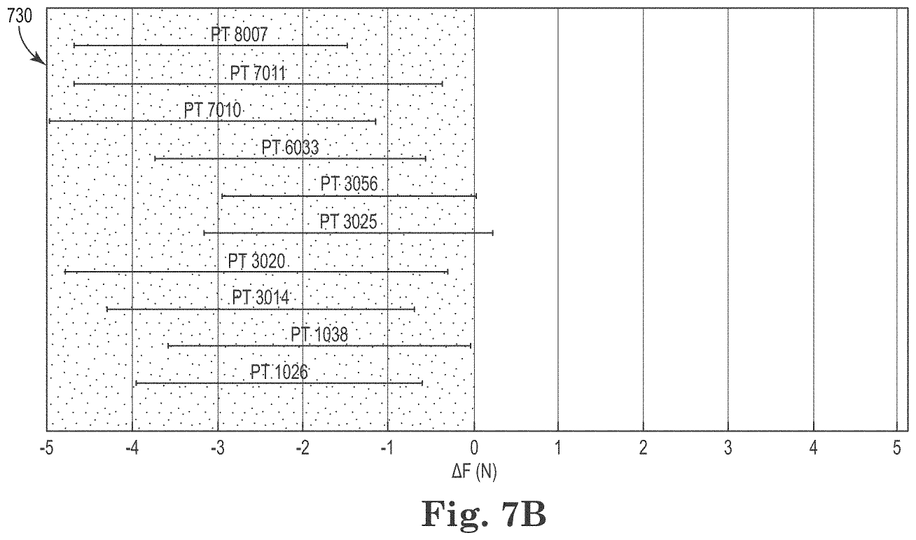

FIGS. 7A and 7B show a device-anatomy force interaction validation against clinical experience. To validate/evaluate the model against clinical cases a back-calculation methodology was developed and implemented (FIG. 7A). This methodology enables us to estimate F_{R1}, F_{R2} etc. components magnitude. In this methodology:
the implant location (i.e. anatomical shape and size of the implant) is estimated using pre-op screening data, as shown at 702. This results in a variability in anatomical size and shape factors within the model. Specifically, the anatomical size 706 and shape 708 associated with all possible implanted scenarios within the LZ and 4 mm above and below it (identified by geometrical fit module).
the back pressures are extracted from intra-operatively post-implant measured PA pressure values at 720 to determine F_{M} 718
the size to COF mapping is done using COF test/FEA, as indicated at 704, to generate F_{R1} 710, and the shape information 708 is used at 714 to generate F_{R2} 714
the distribution of test data for coefficient of friction is used to characterize this factor in the model.

FIG. 7B shows the results 730 of the analysis on 10 implanted clinical cases, which clearly demonstrates consistency of prediction on risk of migration with the clinical outcome. Each error bar represents the predicted probability distribution of migration for a patient.

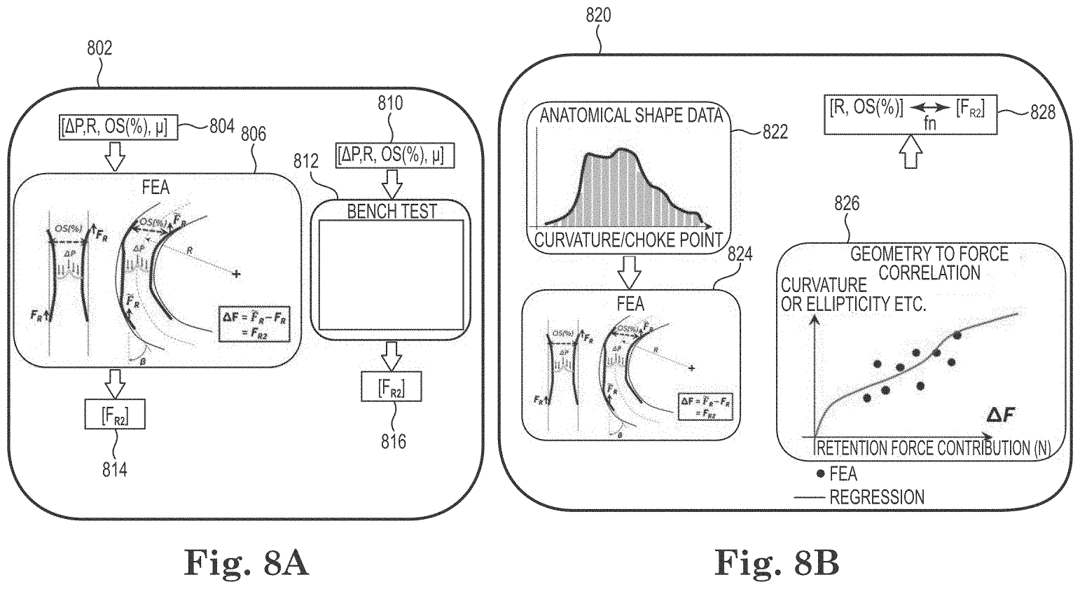

A methodology, heavily relying on physics-based computer simulation, was implemented to quantify the contribution of anatomical shape features to migration resistance force (See FIGS. 8A and 8B). Specifically, a FEA model validated with bench test was used to characterize additional resistance force resulted from each of these shape factors for a range of anatomically relevant curvature and ellipticity values. FIGS. 8A and 8B are diagrams illustrating a schematic representation of a methodology to characterize the contribution of anatomical shape factors (e.g., main PA curvature, ellipticity, etc.) to device retention force according to one embodiment. FIG. 8A shows phase one 802, which involves FEA model development and validation. As shown in FIG. 8A, input information 804 is provided to FEA 806 to determine F_{R2} value 814, and input information 810 is used at bench test 812 to determine F_{R2} value 816 for validation against value 814.

The main PA anatomical curvatures and ellipticities were extracted from pre-op CTA data of a device pivotal dataset using screening fit analysis algorithm. The ellipticity is defined as shown in the Equation I above.

As shown in FIG. 8B, which shows phase two 820 involving F_{R2} estimation, FEA modeling at 824 is one available tool to quantify the contribution of anatomical shape features 822 to resistance force (F_{R2}). In an experiment, the force to cause migration was measured in conduits representative of the shape over a range of sizes by measuring the pressure required to cause movement of the prosthetic device test sample. A static finite element analysis (FEA) was performed to predict the migration backpressure for the modified prosthetic device deployed into the rigid conduit. The conduit geometry, i.e., cross-sectional shape (round or elliptical) and curvature (straight or curved) was investigated. As shown in FIG. 8B, the FEA 824 generates a geometry to force correlation 826, which allows determination of the function 828.

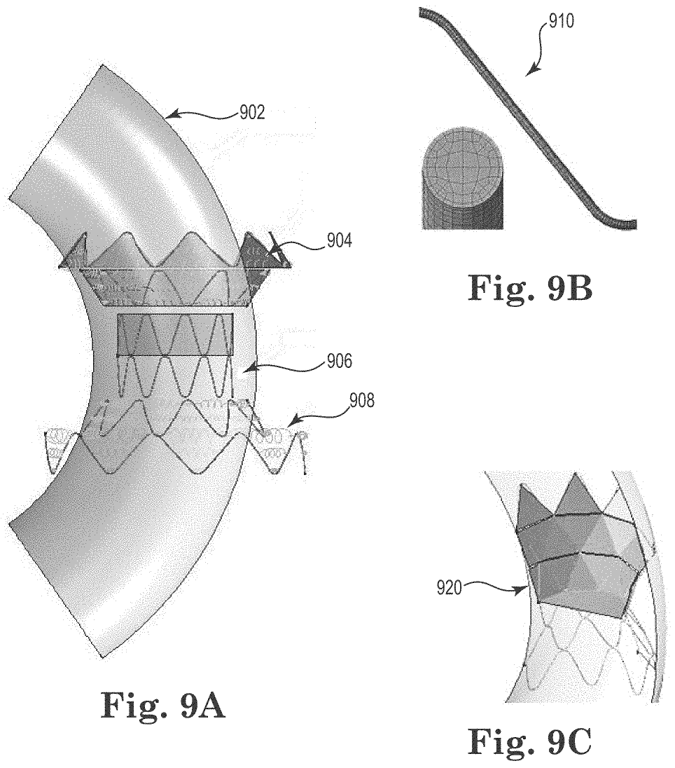

FIGS. 9A-9C are diagrams illustrating a migration FEA model for a curved configuration. FIG. 9A shows a rigid conduit 902, a fabric web for pressure loading 904, a NiTi strut 906, and a fabric with calibrated stiffness 908. FIG. 9B shows a typical strut mesh 910. FIG. 9C shows a frame graft 920 after radially deployed into the conduit.

Results: After the simulation completes, the results were post-processed to determine the backpressure at the onset of migration. Migration backpressure was determined using the following criteria:

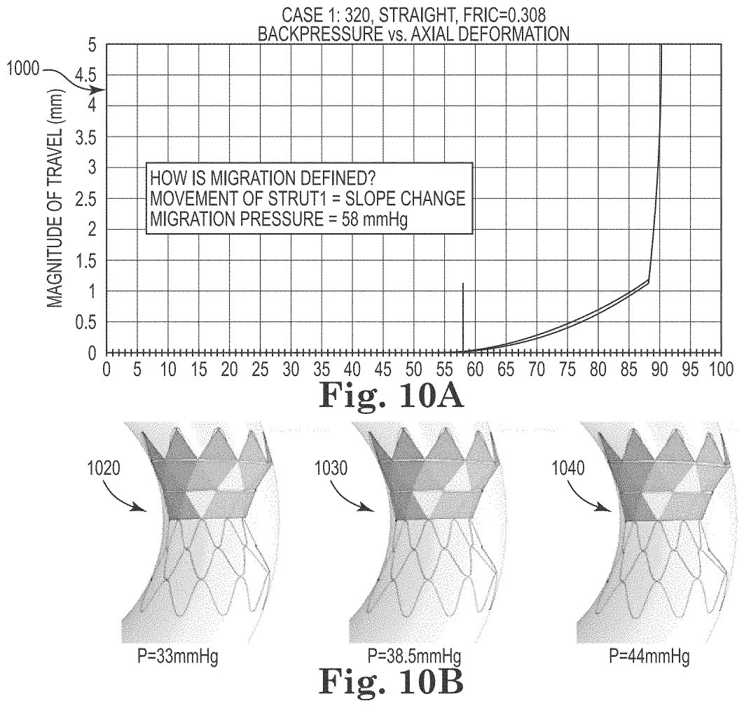

Criteria 1: Magnitude of Travel vs. Backpressure (See FIG. 10A). FIG. 10A is a diagram illustrating a graph 1000 representing FEA migration onset for a first criteria according to one embodiment. The magnitude of deformation at the inflow crowns of strut 1 was graphed as a function of the applied backpressure for each case. The migration backpressure was identified based on a change in slope, as illustrated for Case 1.

Criteria 2: Global Deformation Response (See FIG. 10B). FIG. 10B is a diagram illustrating FEA migration onset for a second criteria according to one embodiment. As confirmation of criteria 1, the global deformation response was monitored. The onset of migration was confirmed visually via animation of the backpressure loading step within the post-processor. This confirmation is required for cases with curvature and ellipticity, because the deformation response is more complicated. Unlike the test environment in which migration is a single dynamic event that cannot be viewed more than once, the simulation allows for repeated views at fine increments of pressure (1.1 mmHg) as the backpressure loading is ramped. Thus, the simulation provides enhanced temporal resolution. FIG. 10B shows three examples 1020, 1030, and 1040 at three different pressures.

The migration backpressure was tabulated by case, and the test results were compared and validated to the migration test results, including a calculation of the percent error between the FEA and the average test result. The migration simulation validates to the test results within an average error of 9%.

Trends predicted by the migration simulation include:

(1) A decrease in migration pressure with an increase in deployed diameter.

(2) An increase in migration pressure due to curvature (compared to the “straight” R=5000 mm baseline) for the small deployed diameter, D=32 mm.

(3) A decrease in migration pressure due to severe curvature, R<60 mm (compared to the “straight” R=5000 mm baseline) for the median deployed diameter, D=35 mm.

(4) A decrease in migration pressure due to nominal curvature, R=60 mm (compared to the “straight” R=5000 mm baseline) for the large deployed diameter, D=38 mm.

(5) An increase in migration pressure with an increase in ellipticity.

The resistive force, F_{R}, due to the effects of curvature and ellipticity (i.e., F_{R2}) was estimated from the increase in migration backpressure compared to the baseline (round, straight) case, as shown in the following Equation III:

F_{R}=ΔP_{migration}*A_{x-section} Equation III

The computed F_{R2} for various corresponding curvature and ellipticity values were fed into a regression model to estimate the transfer functions relating a geometrical factor (e.g., curvature (1/mm)) to retention force (N). These transfer curves or characteristic curves were then implemented on CT-based anatomical distributions to generate corresponding retention force contribution distributions.

These distributions were added to the statistical model to represent the anatomical shape factors, increasing the input source variations for a Monte-Carlo simulator to five (i.e., anatomical size, device variability, coefficient of friction, blood pressure, and anatomical shape). The simulation was repeated for 100,000 iterations and from calculated F_{R1}, F_{R2} and F_{M} values the ΔF=(F_{R1}+F_{R2})−F_{M} distribution is formed, where the area under ΔF>0 indicates the risk associated with migration.

In some examples, the biomechanical interaction analysis module 112 uses a statistical model that is formed to provide an estimate on risk of migration for a prosthetic device. The model is built on a distribution of various devices and anatomical factors such as anatomical size, anatomical shape, physiological pressure, device manufacturing variability (with respect to COF), and device-tissue coefficient of friction. Some examples of the model may be built based on the following assumptions, limitations and considerations:

(1) RV pulse pressure may be used as a surrogate for diastolic back pressure.

(2) The model may or may not directly account for device-tissue embedding, while a certain level of embedding is expected to be present in the test.

(3) The anatomical size extracted from pre-op CTs may be measured by expert imaging analysts.

(4) The post-op anatomical sizes may be estimated using pre-op CT.

(5) The model may or may not take tissue compliance variations into account.

(6) In some embodiments, to characterize anatomical shape factors effect, their effects may be characterized in isolation from other factors, i.e. they may be assumed to be independent parameters in some embodiments.

(7) In some embodiments, the coefficient of friction test may be tested and quantified using a single valve (n=1) repeatedly. Therefore, the two major sources of uncertainty in these embodiments are absence of device manufacturing variations effect, plus device characteristic change due to repeated use.

(8) In some examples of this model, the geometrical factors contributing to retention force may be evaluated independently and superimposed linearly. Therefore, the interplay between the geometrical factors may not be evaluated for some embodiments.

The model may be adjusted and reconstructed for a particular prosthetic device, and evaluated/validated against clinical data. Some examples of this model may: (1) inform the screening process to reduce the risk of migration through a more informed patient selection criteria/approach; and/or (2) provide a tool to evaluate future design concepts.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method, comprising:
receiving, via at least one processor, anatomical measurements of a lumen of a patient;
performing, via the at least one processor, a geometrical fit analysis based on the anatomical measurements to identify potential prostheses to be implanted in the lumen and an optimal implantation landing zone within the lumen for at least one of the potential prostheses, wherein the geometrical fit analysis includes comparing a geometry of the lumen, including anatomical shape factors for the lumen, to geometries of a plurality of candidate prostheses at a plurality of potential implant deployment positions within the lumen;
performing, via the at least one processor, a biomechanical interaction analysis to select one of the identified potential prostheses based on a risk of migration within the lumen of each of the identified potential prostheses; and
outputting, via the at least one processor, an indication of the selected prosthesis and the landing zone for the selected prosthesis.

2. The method of claim 1, wherein the anatomical shape factors include curvature and ellipticity.

3. The method of claim 1, wherein the biomechanical interaction analysis comprises a probabilistic mechanical force analysis.

4. The method of claim 3, wherein the force analysis comprises a comparison between a migration force based on physiological pressure and a resistance force that resists migration.

5. The method of claim 4, wherein the resistance force includes at least one of a friction force component based on anatomical size and prosthesis specifications, an anatomical barrier force component based on anatomical shape factors, and a prosthesis-tissue embedding force component based on a biomechanical interaction between prosthesis and tissue.

6. The method of claim 3, wherein the force analysis comprises a finite element analysis.

7. The method of claim 1, and further comprising:
displaying the landing zone on a simulated intraoperative fluoroscopic image.

8. The method of claim 1, and further comprising:
displaying the landing zone on a live intraoperative fluoroscopic image for intraoperative visual guidance.

9. The method of claim 1, wherein the prostheses are prosthetic heart valves.

10. The method of claim 1, wherein the landing zone is within a pulmonary artery.

11. A method of identifying a prosthesis for implantation and a landing zone for implantation of the prosthesis within a patient's anatomy at an implantation site, the method comprising:
receiving, via at least one processor, a three-dimensional model of the implantation site;
analyzing, via the at least one processor, for each of a plurality of potential prostheses, a plurality of potential prosthesis deployment positions and axis orientations relative to the three-dimensional model;
identifying, via the at least one processor, the prosthesis for implantation from the plurality of potential prostheses based on the analyzing;
identifying, via the at least one processor, a landing zone at the implantation site for the identified prosthesis; and
generating, via the at least one processor, a display illustrating the landing zone in a preoperative image.

12. The method of claim 11, wherein the analyzing comprises a probabilistic mechanical force analysis.

13. The method of claim 12, wherein the force analysis involves a comparison between a migration force and a resistance force that resists migration.

14. The method of claim 12, wherein the force analysis comprises a finite element analysis.

15. The method of claim 11, wherein the prostheses are prosthetic heart valves.

16. A non-transitory computer-readable storage medium storing instructions that, when executed by a processor, cause the processor to:
perform a geometrical fit analysis based on anatomical measurements at a prosthesis implant site of a patient to identify potential prostheses to be implanted at the implant site, wherein the geometrical fit analysis includes comparing an anatomical geometry at the implant site to geometries of a plurality of candidate prostheses at a plurality of potential implant deployment positions at the implant site;
perform a probabilistic mechanical force analysis to determine a risk of failure of each of the identified potential prostheses; and
output a recommendation identifying one of the potential prostheses based on the probabilistic mechanical force analysis.

17. The non-transitory computer-readable storage medium of claim 16, and further storing instructions that, when executed by the processor, cause the processor to:
generate a display of the recommended prosthesis at a recommended deployment position at the implant site to facilitate implantation of the prosthesis in the patient.

18. The non-transitory computer-readable storage medium of claim 16, wherein the force analysis comprises a comparison between a migration force tending to cause prosthesis failure and a resistance force that resists the migration force.

19. An electronic prosthesis analysis tool, comprising:
a memory to store a plurality of different design concepts for a prosthesis; and
a processor to perform a probabilistic mechanical force analysis on the plurality of different design concepts to determine prosthesis failure risk information for each of the design concepts and identify a best one of the design concepts based at least in part on the prosthesis failure risk information.

20. The electronic prosthesis analysis tool of claim 19, wherein the prosthesis is a prosthetic heart valve.
